(19) United States
(12) Patent Application Publication
(10) Pub. No.: US 2012/0289878 A1
(43) Pub. Date: Nov. 15, 2012

(54) LOW PROFILE HIP ORTHOSIS

(76) Inventors: Shannon R. Schwenn, Deltona, FL (US); Gregory Joseph Paaske, Eustis, FL (US); Philip J. Dalmada Remedios, Dunedin, FL (US); Robert Sean Hagen, Dunedin, FL (US); Patrick Burgess, Dunedin, FL (US)

(21) Appl. No.: 13/511,114

(22) PCT Filed: Nov. 23, 2010
(86) PCT No.: PCT/US10/57889

§ 371 Date: May 21, 2012

Related U.S. Application Data
(60) Provisional application No. 61/264,168, filed on Nov. 24, 2009.

(51) Int. Cl. A61F 5/00

(52) U.S. Cl. 602/23

(57) ABSTRACT

An orthosis for supporting a body portion of a user, for example, a hip joint or spine that has been compromised, can utilize an adjustable extension unit that can be appropriately secured, for example, to anchor locations on the body between the waist and a thigh of the patient. A body support unit with a compliable support surface can conform to a body surface part of the user, such as the leg, while being movably mounted across the adjustable extension unit between the anchor locations. The adjustable extension unit can include a pair of telescoping elongated members that are relatively movable and both the body support unit and the collective length of the telescoping elongated members can be adjusted and fastened at a desired location by activation of a control member.

### CROSS-REFERENCE TO RELATED APPLICATION

The present application claims priority from U.S. Provisional Application Ser. No. 61/264,168 filed on Nov. 24, 2009.

### BACKGROUND OF THE INVENTION

1. Field of the Invention

The present invention is directed to an orthosis with an adjustable body support portion for conforming to a patient's configuration such as a low profile hip orthosis for post-surgery treatment of a patient and more particularly to a patient who has had a hip arthroscopy procedure.

2. Description of Related Art

An orthosis which can conform to a particular body configuration has required a customized orthosis made from body measurements of a patient. However, medical and insurance costs have significantly increased and the provisions of pre-manufactured orthosis that can be adjusted by an orthotist have become more popular.

An example is an orthotic to provide support for the body after replacement of a hip joint or hip arthroplasty, which is a surgical procedure in which a hip joint is replaced by a prosthetic implant. A femoral component can be replaced with a metallic or ceramic artificial femoral head where ceramic bearing surfaces are frequently employed to address any wear issue and relieve possible problems from osteolysis that can occur from acrylic bone cement and wear debris.

Risks that are involved in the surgery are a potential for blood clots in the legs, infections, and nerve damage. Generally it is desirable to have the patient start moving and walking as soon as possible to prevent complications. Postoperative hip orthoses have been known to reduce the incidences of dislocation in both patients who have undergone a primary hip replacement and also patients that have required a subsequent hip revision. Reference can be made to a clinical report, Orthotic Management of Hip Dislocation using a Newport™ hip orthosis by Dulcey Lima.

Orthopedic hip configurations have been utilized for various purposes such as a leg abductor to resist muscular contractions as shown in U.S. Pat. No. 5,814,001. U.S. Pat. No. 6,589,195 and U.S. Pat. No. 7,048,707 are examples of hip orthoses for permitting an orthotic management when the hip joint has been compromised with the capability of controlling flexion and extension internal and external rotation adduction and abduction. In such an environment, the hip joint is a synovial ball and socket joint that generally consists of an articulation of the spherical head of the femur with a cup like shape of the acetabulum. The acetabular labrum attaches to a bony rim of the acetabulum and cups around the head of the femur to hold it firmly in place. Various ligaments add strength to the articulation of the hip joint and a large number of muscles act on the hip joint. The gluteus medius is primarily associated with abduction. The anterior fibers assist with flexion and internal rotation. Posterior fibers assist with the extension and external rotation. These respective muscle groups stabilize the pelvis during a single leg support.

However, these muscle groups can be compromised when surgical procedures are performed at the hip joint such as a hip replacement surgery. A significant problem that occurs when a hip joint has been compromised, is the possibility of subsequent dislocation of the hip joint. Thus, the femoral head can be driven out of the acetabulum. For example, the hip can be most susceptible to posterior dislocation when it is flexed past 90°, internally rotated and abducted. These movements can occur in everyday life, such as by sitting on a low chair and leaning forward while putting weight on an affected hip joint and internally rotating when coming to a standing position. Thus, common activities of daily living, specifically excessive hip flexion with loaded extremity and internal rotation on the affected side can cause dislocation. Anterior dislocation can also occur when a hip is externally rotated, abducted and flexed. For example, if there is an external force such as the knee being hit or subject to an accidental contact with an object, the neck of the femur or the greater trochanter levers the femur out of the acetabulum. Thus, an orthosis must be able to effectively control the limits of extension rotation in a patient who has experienced an anterior dislocation.

Additionally, as the population becomes older, there are more occasions for the treatment of hip disorders and there is still a need to improve the function of such orthoses and their component parts in the medical field in an economical manner while addressing a comfort level for the patient to encourage maximum prolonged usage.

Efforts have also been made to encourage early mobility of a patient after a hip surgery and it is desirable to enable the patient to easily don a hip orthosis with appropriate compression with a lightweight structure. It is still necessary for the hip orthosis to provide adequate rigidity to stabilize the hip with preferably a configuration that would encourage prolonged use by the patient. U.S. Pat. No. 7,473,235 is an example of a lightweight prophylactic hip orthosis. U.S. Pat. No. 7,597,672 discloses a hip orthosis with a rotable leaf spring for the user to actuate after donning the hip orthosis.

Orthopedic surgeons have now developed new techniques for repairing the hip joint that can often provide pain relief and postpone or eliminate the need for total joint replacement. This procedure, hip arthroscopy, utilizes an extremely small and long camera called an arthroscope that allows the surgeon to visualize the inside of the joint without the need for an open surgical procedure. Traction is applied to the foot to distract the hip joint so that sufficient space can be created between the hip joint surfaces to insert specialized instruments. Arthroscopy uses a variety of “shavers” used to debride the frayed labrum or cartilage, and uses specialized tools to reconnect and repair soft tissue, and incorporates fluoroscopy to determine the precise location of the surgical instruments. Fluid is inserted through the hip joint to both clear debris and improve visualization for the surgeon. Hip arthroscopy is a less invasive procedure with significantly less soft tissue trauma than the traditional open surgical procedures such as total hip replacement (arthroplasty).

Hip arthroscopy can treat labral tears in the labrum cuff of thick tissue that surrounds and supports the hip joint. Additionally, if pieces of cartilage form within the hip joint they can be removed by lavage and rough edges in the hip joint can be scraped and smoothed. Additionally, the synovial lining of the hip joint can be addressed if inflamed.

While a hip arthroscopy procedure can frequently be performed as an outpatient procedure without spending days in a hospital, it is still necessary to rehabilitate the muscles around the joint to make them stronger to assist the weight-bearing surfaces within the hip joint and increase the range of motion of the hip joint. The proper use of exercise in the recovery process encourages healing and failure to exercise and move on the hip joint can even lead to permanent disability.

There is still a need in the medical industry to provide a relatively lightweight and low profile orthosis that can be easily adjusted to meet the needs of the patient, for example in the post treatment of hip arthroscopy patients that can be readily adjustable by the orthotist for the particular patient while being lightweight and of a low profile to encourage prolonged use by the patient and effectively protect any compromised hip joint.

### SUMMARY OF THE INVENTION

The present invention provides an orthosis for providing a particular support of a body portion of the user such as a body support unit with a support surface for conforming to the body surface of a part of the user. An adjustable extension unit can be slidably connected to the body support unit and releasably connected to the user, for example, at the waist and leg of the user for a hip brace, or at the waist and shoulders of the user for a spinal brace.

The adjustable extension unit can include a pair of telescoping elongated members that are relatively movable to adjust both the length of the adjustable extension unit and the particular desired position of the body support unit on a particular user. The body support unit can have a base portion for slidable contact with the adjustable extension unit with a support housing mounting a moveable control member that locks the body support unit and the pair of telescoping elongated members to the fixed position.

The support housing member can be connected to the body support unit through openings in the pair of telescoping elongated members to thereby permit a movable control member and to be positioned on the support housing on one side of the adjustable extension unit and the body support unit with a slidable base portion positioned on the other side so that the movable control member can compress the respective components together to a locked position or can release the respective components for relative adjustable movement.

The movable control member, when moved to a locked position, forces the base portion into a locking pressurized contact with the adjustable extension unit to prevent relative movement of the telescoping elongated members while locking the body support unit in a predetermined position on the adjustable extension unit.

The connector member can be a fastener member such as a bolt, screw or rod that can be secured to the movable control member and activated to compress the telescoping elongated members in a lock mode of operation and release the telescoping elongated members in a release mode of operation when the movable control member is moved from the lock mode to a release mode. The movable control member can be, for example, a rotable knob connected to a worm gear, a pivotal lever member movably connected to support housing with a cam surface to move the fastener member or other configurations to provide a relatively small movement that can be easily locked and released.

A low profile orthosis is designed to be easily attached and released from a patient with a strong but lightweight configuration. In an example of a hip orthosis, a hip engaging unit includes a malleable hip member or band for conforming to the waist of the user about the user's pelvis with a closure unit for securing the hip engaging unit on the waist. The malleable hip member provides an anchor point so that a lateral force can be applied to the user's pelvis opposite a surgical site.

A thigh engaging unit, including a malleable thigh member, is designed to conform to the thigh of the user to engage a femur of the user on the surgical site side and on the distal side of the femur. Thus, a lateral force can also be applied to the distal side of the femur to complement the lateral force applied by the hip engaging unit. A lightweight adjustable extension unit can be connected or anchored to the hip engaging unit and to the thigh engaging unit and can be adjustable to accommodate the particular size or length of the leg appendage of the user with the activation of a simple control member.

Located below the surgical site and slidably connected to the adjustable extension unit at a position between the hip engaging unit and the thigh engaging unit is a thigh support unit configured to interface with the leg to exert a force on the leg to push the head of the user's femur into the acetabulum of the user. Thus, a counter lateral force can be created to press the femur of the user whereby a force is translated through the femur and pelvis to push the head of the femur into the acetabulum of the user when the hip engaging unit and the thigh engaging unit are fastened to the user.

The adjustable extension unit includes a pair of elongated members that overlap and are mounted for telescoping movement. The elongated members can be flat bars with appropriate slots to permit fasteners to secure them together at the appropriate length.

Alternatively, the adjustable extension elongated members can be an extruded lightweight pair of components of an approximately W cross-sectional shape, having an outer extension member with side rails or ledges to capture the complementary configured interior extension member. The body support unit such as a thigh support unit can be slidably mounted to encircle the extension members with a movable control member such as a camming lock member to releasably lock and hold the thigh support unit at a predetermined location along the extension members while fastening the respective extension members together.

The thigh support member can include a flexible concave paddle member configured to conform to and interface with a body surface part of a user such as an outside configuration of the leg below the surgical site to accommodate the applied force. The concave paddle member can have a central base, for example of a resin material such as nylon, for providing a sliding contact with the adjustable extension unit which is laterally offset from the central base portion that is configured to replicate the surface of the adjacent adjustable extension unit. A pair of elongated side openings in the paddle member to define perimeter outer edge panels for relative movement in relation to the central base to enable support adjustment to conform to the surface configuration of the user's leg. To provide a lightweight configuration and to assist in air circulation, appropriate apertures or holes, for example of an oval configuration, can be provided in the outer edge panels. A compressed foam pad can be mounted over the concave paddle member to provide a relatively softer contact surface to the user's leg.

Interconnecting the central base with a support housing position on the other side of the adjustable extension unit is a support housing member mounting a movable control member such as a pivotal lever member, that is operatively connected to the central base, for example by a connector rod or bolt member that can extend through elongated slots in the adjustable extension unit for direct connection to a cylinder or barrel nut that can provide a pivotal shaft for rotating a camming surface on the end of the pivotal lever for pulling the central base into frictional contact with the adjustable extension unit to lock the thigh support member at an appropriate position along the adjustable extension unit. Thus, in this embodiment of the invention, a control member in the form, for example, of a pivotal lock or other configuration, provides a dual function of setting the appropriate length of the adjustable extension unit while also locking the thigh support member so that the orthotist can provide a simple adjustment to match the particular size of the patient or user.

The thigh member can have a U-shaped configuration for partially encircling a user's thigh at a position above the user's knee joint with a flexible and adjustable closure member connected to a female receptacle member and a male prong locking member for setting and releasing the closure member at a predetermined fixed distance. The U-shaped malleable thigh member can be formed of either plastic or aluminum to permit subjective adjustment to the shape of the individual user's thigh.

A hip joint member can interconnect the adjustable extension unit with the malleable hip member to control both flexion and extension movement of the user's leg and an abduction adjustment. Compressed foam pads can be appropriately positioned on both the thigh support member of the hip engaging unit and the thigh engaging unit to provide comfort to the user. The malleable hip member can conform to the waist of the user with a series of aligned keyhole slots to permit an adjustment with a connecting anchor member capable of being held within a keyhole slot so that an adjustable web can be utilized to provide a closure unit, for example with nap and hook material to a particular dimension of the user.

### BRIEF DESCRIPTION OF THE DRAWINGS

The objects and features of the present invention, which are believed to be novel, are set forth with particularity in the appended claims. The present invention, both as to its organization and manner of operation, together with further objects and advantages, may best be understood by reference to the following description, taken in connection with the accompanying drawings.

FIG. 1 is an elevated view of a first embodiment of the low profile orthosis of the present invention disclosing its relationship with the conical skeletal structure of a user;

FIG. 2 is a perspective view of the first embodiment;

FIG. 3 is a perspective view of the first embodiment without cushioning pads to view the relationship of a malleable hip member and a malleable thigh member;

FIG. 4 is side view of FIG. 3;

FIG. 5 is a perspective view of a second embodiment of a low profile hip orthosis of the present invention;

FIG. 6 is an exploded view of the thigh support unit and adjustable extension unit of the second embodiment;

FIG. 7 is a partial cross-sectional view of a portion of the thigh support unit and adjustable extension unit in an unlocked slidable configuration;

FIG. 8 is a partial cross-sectional view of the thigh support unit and adjustable extension unit in a locked or clamped fixed position with a thigh support unit and length of the adjustable extension unit;

FIG. 9 is a cross-sectional view of the thigh support unit and adjustable extension unit; and

FIG. 10 is a perspective exploded view of the thigh support unit.

### DETAILED DESCRIPTION OF THE PREFERRED EMBODIMENTS

Reference will now be made in detail to the preferred embodiments of the invention which set forth the best modes contemplated to carry out the invention, examples of which are illustrated in the accompanying drawings. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention as defined by the appended claims. Furthermore, in the following detailed description of the present invention, numerous specific details are set forth in order to provide a thorough understanding of the present invention. However, it will be obvious to one of ordinary skill in the art that the present invention may be practiced without these specific details. In other instances, well known methods, procedures and components have not been described in detail as not to unnecessarily obscure aspects of the present invention.

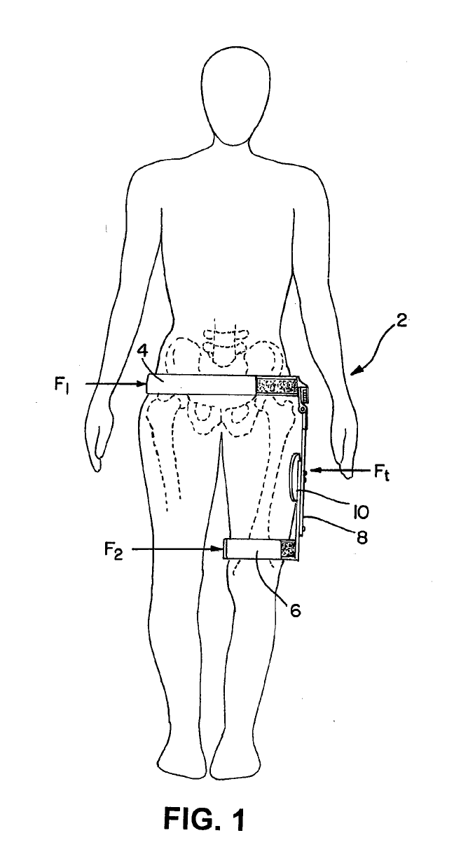

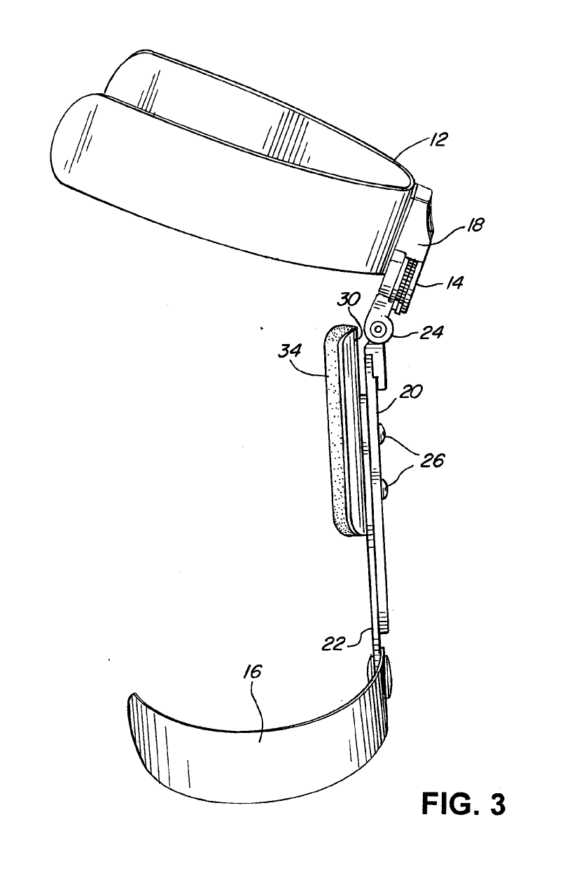

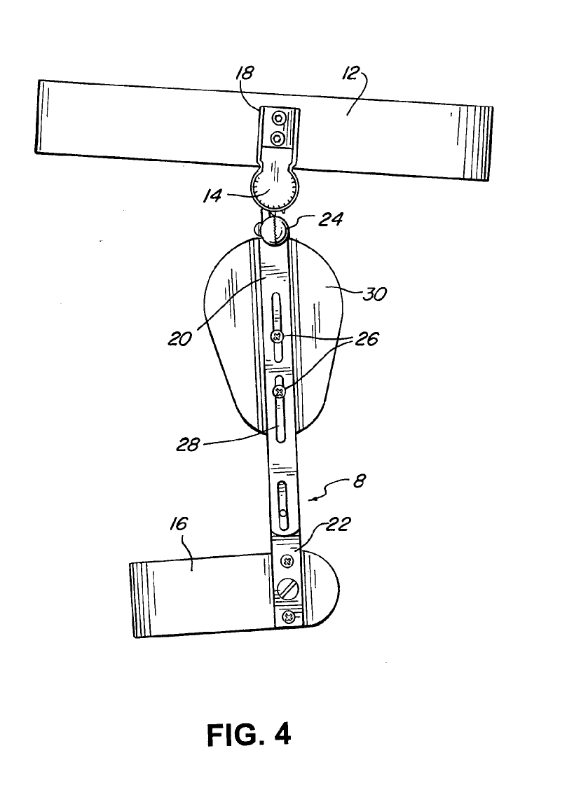

Referring to FIGS. 1-4, a first embodiment of the present invention is disclosed where a low profile orthosis 2 basically includes a hip engaging unit 4, a thigh engaging unit 6, an adjustable extension unit 8 for interconnecting the hip engaging unit 4 and the thigh engaging unit 6 and a thigh support unit 10 adjustably mounted for movement relative to the adjustable extension unit 8. As shown in FIG. 1, the hip engaging unit 4 and the thigh engaging unit 6 can be adjustably mounted to respectively the waist or pelvic section of a user and the lower thigh of the user. Covering material and appropriate padding material is provided between the operative components and the surface of a user. Additionally appropriate cushioning such as a foam compression pad and covering can also be provided on the thigh support unit 10.

The low profile hip orthosis 2 includes a pelvic band formed for example, of a malleable aluminum or plastic material having a length to extend the majority of the distance around the waist of the user with a relatively narrow width. For example, the width, except for the attachment of a hip hinge 14, can be the same width or can be extended in width to prevent dislocation during movement of the user. Thus, the malleable pelvic band 12 can be approximately 23 inches in length with a width of approximately 2 inches and a thickness of approximately 0.062 inches of aluminum which can be altered to adjust the particular dimensions of the waist of the patient. The pelvic band 12 can be longer on the posterior side of the user than on the anterior in order to increase stability of the base in relationship to the pelvis. The aluminum can be bent or cut by an orthotist in subjectively configuring the low profile hip orthosis to a particular patient to secure a firm anchoring location on the waist of the user.

Collectively, these components are relatively lightweight and function to provide a controlled movement of the leg during walking and sitting with an application of a force, Fₜ that is counter balanced by forces F₁ on the distal side of the hip engaging unit 4 and the force F₂ on the distal side of the thigh engaging unit.

As can be appreciated, other lightweight metals, polymers and composites can also be utilized as long as they permit the hip engaging unit 4 to conform to the waist of a user about the user's pelvis to enable the lateral force F₁ be applied to the user's pelvis opposite a surgical site. Additionally, the same type of metals, polymers and composites can also be used to permit a malleable thigh member 16, of approximately 12 inches in length of aluminum with a thickness of 0.062 inches and a width of 2 inches, to conform to the user's lower thigh above the knee and provide an anchoring force F₂ wherein the respective forces F₁ and F₂ enable the thigh support unit 10 to provide a counter force Fₜ.

Our improvements are of particular value as a remedial hip orthosis for patients who have undergone hip arthroscopy surgery and require a relatively lightweight and low profile hip orthosis for a post treatment. The hip orthosis can be readily adjusted by the orthotist to provide minimal structure to thereby encourage prolonged use by the patient and to effectively protect the compromised hip joint while encouraging the proper use of exercise in the recovery process to facilitate both the healing and strengthening of the muscles that may have been compromised during the surgery. Thus, the patient is encouraged to immediately start moving and walking to avoid post surgery complications while reducing the incidence of any dislocation in the hip joint. The lateral force Fₜ and the limiting movements with a hip hinge 14 that is adjustable to control flexion and extension and limit abduction, enable the user to be encouraged to safely engaged in prescribed physical rehabilitation while having the femur head of the hip joint being biased in a lateral direction for seating in the acetabulum.

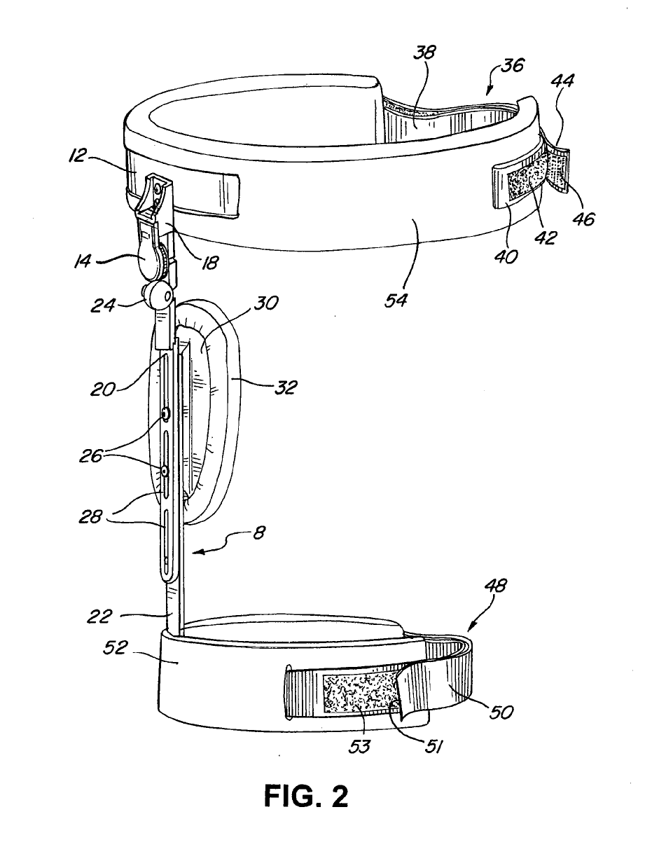

Referring to FIG. 2, the hip hinge 14 is anchored onto the mounting bracket 18 which is fastened by appropriate fasteners directly on the pelvic band 12. In turn, the hip hinge 14 can be connected by an adjustable ball joint member 24 where respective spherical halves with interfacing teeth can be adjustably fixed by the tightening of a screw through the center of the ball joint to provide an appropriate abduction or offset from the side of the user. As shown in FIG. 2, the ball joint member 24 is in turn connected to an exterior elongated narrow plate 20.

An interior elongated narrow plate or bar 22 is fastened to the thigh band 16. The extension bars 20 and 22 are fashioned from a 0.170 inch thick engineering grade aluminum and are each approximately 9 inches long. Two or more fasteners 26 can be mounted through appropriate slots in each of the exterior and interior elongated plates 20 and 22 to permit an adjustable mounting to set the length of the adjustable extension unit 8. As can be seen in FIG. 2, one of the fasteners 26 can also be utilized to adjustably attach a thigh paddle 30, for example of an oval or oblong shape formed from a punched or extruded aluminum plate of a size to be adjustable by an orthosis to accommodate people of different thigh shapes. The thigh paddle 30 is appropriately adjustable in position along the adjustable extension unit 8 and can have a concave configuration to simulate the contour of the hip with a centerline groove or valley to receive fasteners without contacting the user. A thigh cover member 32 can include a perimeter of an elastic material to permit the removal of the thigh cover member 32 for washing purposes. A compressed foam pad, not shown, can be used to cover the thigh paddle member for cushioning purposes with the user.

A waist closure unit 36 can be appropriately secured to the hip engaging unit with a webbed flexible band 38 with one end of the web 40 having a nap material while another end of the web 44 has hook material 46 for example, of the type known as Velcro™ that can be appropriately adhered so that the band 8 will be adjustably extended through a loop attached to one side of the band 12 to permit the user to attach an anchor end 44 of the band 8 with the hook material 46 on the nap material 42.

Likewise, a thigh closure unit 48 also includes a flexible web band 50 to act as a fastening member having Velcro™ hooks 51 at one end for permitting a tightening and adjustment of the thigh band 16 to the particular dimensions and size of the patient. As can be readily appreciated, the particular choice of the length of the thigh bands, pelvic bands and thigh paddles can vary depending upon the size of the patient. Additionally, a pelvic band sleeve 54, as can be seen from FIG. 2, is designed to encapsulate the respective pelvic band 12. Also thigh sleeve 52 can cover the thigh band 16 by permitting a sliding location over the respective U or C-shaped configurations. The thigh closure unit 48 can also have an appropriate loop buckle with the Velcro™ type hook 51 and nap material 53 for securement purposes. It is also possible, although not shown in the drawings, to include a retainer strap with an open aperture to permit a thumb to pull and tighten the straps on the patient. Both the thigh cover member 52 and the respective soft goods cover member 54 for the waist or hip engaging unit and for the thigh engaging unit 30 can have resilient material positioned in areas that contact the material covering the patient's skin to have a taut configuration in the areas that contact the patient's skin while permitting the respective soft goods to be removed and washed.

It should be appreciated that the thigh paddle 30 could be further supplemented to provide an adjustment of the pressure to be applied to the femur by using mechanical members such as a screw fastener, an air bladder or similar devices known in the orthopedic field. The thigh band 16 can be adjusted through a thigh band pivot (not shown) so that its angular disposition relative to its support plate 22 can be adjusted to the size of the thigh of the particular user. The thigh band 16 can be made of aluminum or other metallic element that can be deformed by an orthotist or from a plastic resin.

As seen in FIG. 1, the low profile hip orthosis 2 of the present invention provides a three point pressure system over the proximal thigh for stabilizing the trochanter into the acetabular socket, thereby preventing a dislocation while maintaining comfort to ensure the extended use by the user. Thus, a prophylactic orthosis is provided to assist the recovery period of the user while maintaining a limited size orthosis and avoiding discomfort to the patient while firmly providing the necessary limitations and protection during the recovery period while encouraging mobility. As a result of this three point pressure system, our low profile hip orthosis 2 provides pressure forces to three separate points, namely a lateral pelvis position, F₁ opposite the surgical site, the femur, Fₜ, on the side of the surgical site, and the distal femur position, F₂, opposite the surgical site. Overall, these respective forces translate through the femur and pelvis, thereby stabilizing the head of the femur into the pelvic socket (acetabulum) during physical activity.

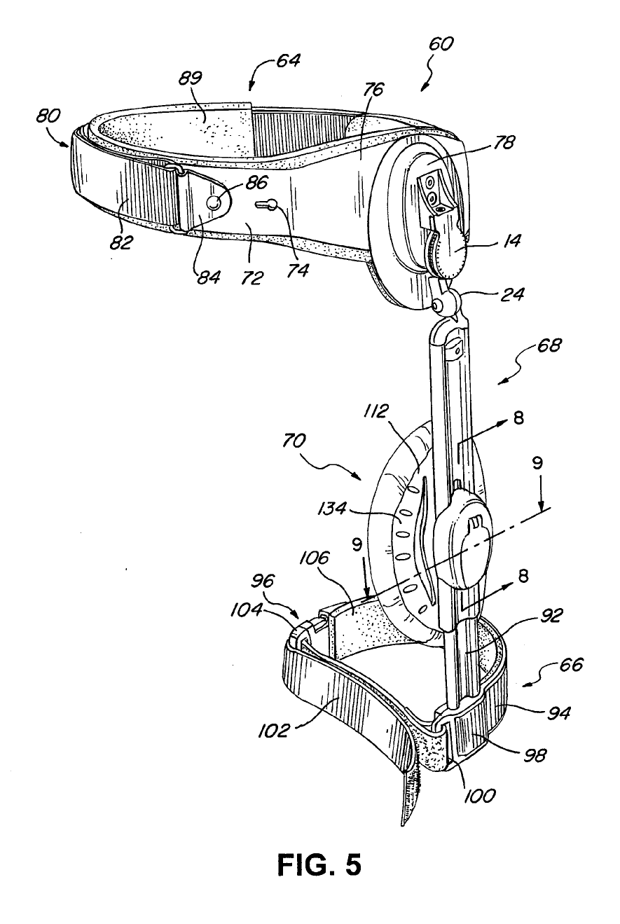

FIGS. 5-10 disclose a second embodiment of the present invention. This modified form of a low profile orthosis 60 also includes a hip engaging unit 64, a thigh engaging unit 66, an adjustable extension unit 68, and a thigh support unit 70.

The hip engaging unit 64 includes a pelvic band 72, for example of plastic, composite or aluminum material with basically the same thickness as mentioned with regards to the embodiment of FIG. 1, with a series of keyholes 74 and a wider hip band area 76 to provide a greater stability in preventing any rotation of the pelvic band 72. Adjacent the attachment of a hip hinge or joint 14 to the hip engaging unit 64 is a large mounting housing 78 of an oval concave configuration to conform, vertically, along a side of the user's hip to provide an enlarged anchor position to prevent any relative rotation or movement of the hip engaging unit 64 when appropriately fastened to the user.

A removable closure unit 80 includes an elongated flexible web band 82 passing through a loop anchor plate 84 having a bolt 86 with a grooved pulley (not shown) mounted to the anchor plate 84 and of a dimension to extend through a large diameter portion of the keyhole 74 and to be captured by a smaller dimension of the keyhole 74 by an interface with the grooved pulley to thereby provide an adjustable connection for the closure unit 80.

The flexible web band 82 includes a hook and nap material at one end that can be mounted so that the hook material is adjustably attached to a swatch of nap material fastened directly to the pelvic band 72 adjacent one end of an opening portion of the C configuration. This provides an exterior over coating of nap material on the web band 82 on the other side of the fastened hook material. The other end of the pelvic web band 82 can have a glued swath of loop material to enable the flexible web band 82 to pass through the loop and be subsequently connected to the loop anchor plate 84 to permit an adjustable closure arrangement on the patient.

The adjustable extension unit 68 is connected to an adjustable ball joint 24 and subsequently attached to an exterior telescoping member 88 which can be formed, for example, of an aluminum extrusion with inwardly projecting side rails 90 to form basically a cross-sectional W shape. An interior telescoping member 92 is slidably mounted with a conforming W shape to be captured by the inner side rails 90. The interior telescoping member 92 in turn is anchored to the thigh engaging unit 66. Thus the adjustable extension unit 68 is fastened to the waist and thigh of the user.

The thigh engaging unit 66 can comprise a one piece injection molded thigh strap member 94 having at one end a female coupling member 96 with open side apertures while the other side of the thigh strap member 94 can have a molded anchor portion 98 with an open cavity of roughly a W configuration to receive the interior telescoping member 92 so that it can be anchored, for example by a pair of bolts or screws to fasten the interior telescoping member 92. This enlarged end piece or extension coupler 98 can also include an integrally molded loop 100 for receiving a closure strap 102 with appropriate nap and hook material to permit an adjustment and closure to a desired length for the closure strap 102. The strap 102 can also capture a prong-shaped male coupler member 104 of a configuration so that the prongs will fit within the female coupler 96 and can be depressed by a thumb and finger of the user to permit the prongs to be accessed through the aperture in the female coupler 96 for releasing the male coupling member 104.

A resilient compression pad 106 similar to the pelvic compression pad 89 can be connected by adhesive to the inside surface of the thigh strap member 94. As can be appreciated, the thigh strap member 94 could alternatively take the configuration of a malleable aluminum thigh band disclosed in embodiment 1. Either structure can provide a stable anchor to the thigh. The thigh strap member 94 is shown with basically a C shape of approximately 12 inches long by 2 inches wide. The C-shaped band 94 rests atop a thigh when properly worn since the forces which are known to cause dislocation, such as those experienced when sitting or crouching are restrained at this location. It is unnecessary to hold the back of the thigh of the patient in this fashion and accordingly, the closure strap 102 can tighten and bend the C-shaped band to serve its appropriate function. As can be seen from FIG. 5, the thigh strap member 94 is positioned at a distal aspect of the femur above, approximately, the knee. See also FIG. 1.

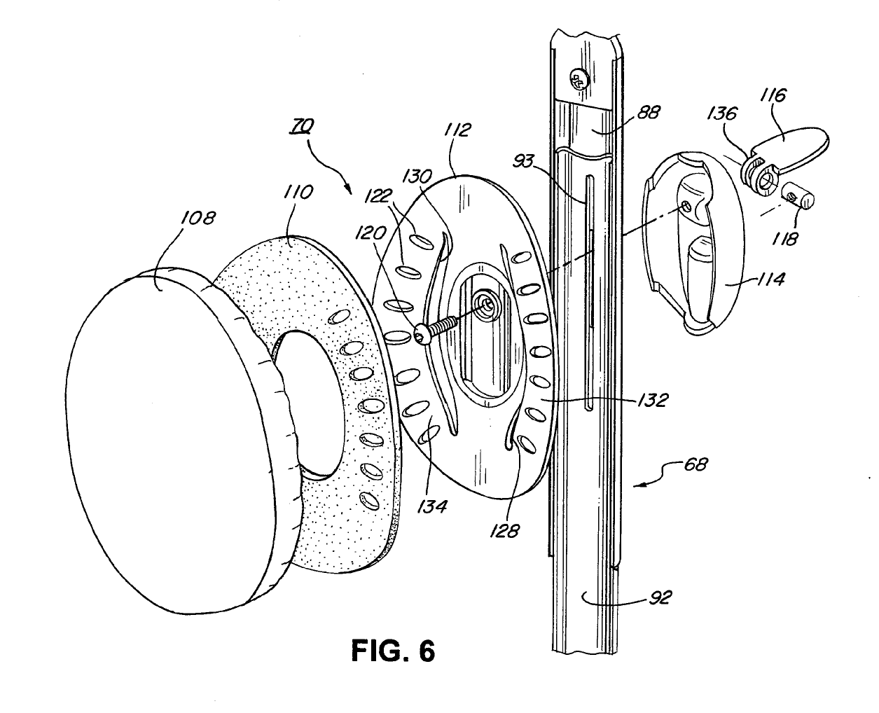

FIG. 6 discloses an exploded view of a body support unit in the form of a thigh support unit 70 and includes, respectively, a removable and washable cover member 108. A resilient compression pad 110, a relatively flexible concave paddle member 112 formed from plastic material such as nylon or polypropylene. The adjustable extension unit 68 is sandwiched between the paddle member 112 and a plastic resin support housing 114 with a movable control member 116 mounted in the support housing by a cylinder barrel nut 118 that engages a bolt member 120.

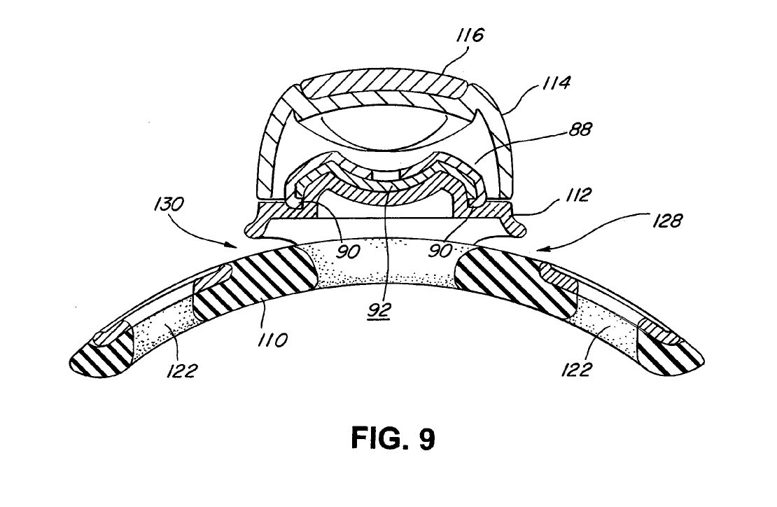

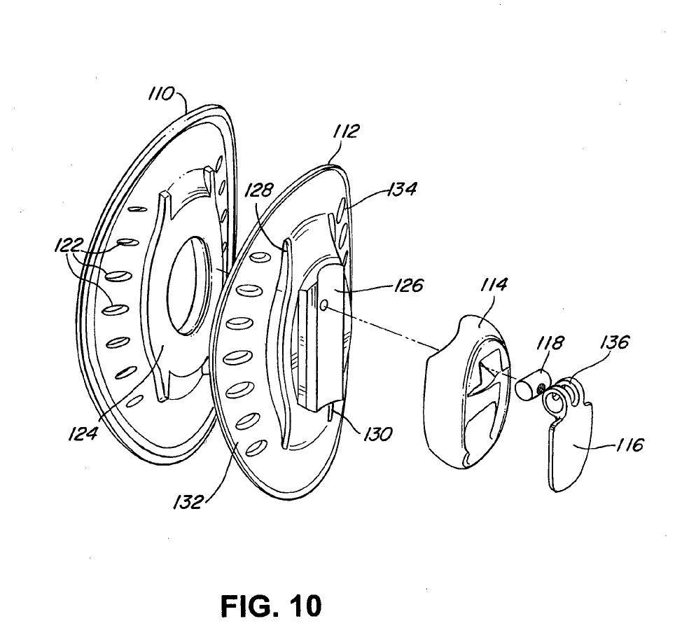

Reference can also be made to FIGS. 9 and 10 for different perspective views of the thigh support unit 70th a compression pad 110 configured with a series of oval apertures 122 in a central base 124 of the same molded material constituting the compression pad 106 and configured to complement the interior vertical portion of a concave paddle member 112. The compression pad 110 can be adhered by an adhesive to the surface of the paddle member 112 that also has corresponding apertures that can be aligned to provide ventilation and to lighten the structure.

The paddle member 112 has a central base 126 of resin material capable of sliding across the surface of the interior telescoping member 92 and the side rails 90 as shown in FIG. 9. The configuration of the surface of the central base 126 also has a complementary W like configuration. As can be further seen in FIG. 9, the support housing 114 extends over the adjustable extension unit 68. The elongated slots or curved opening apertures 128 and 130 create a pair of perimeter bridge plates 132 and 134 to provide flexibility to accommodate different sized thighs while providing a resilient force for pushing the femur.

As can be appreciated with this embodiment of the present invention, a single bolt 120 can extend through respective elongated slots 93 in the adjustable extension unit to interconnect the concave paddle member 112 so that its central base 126 slides in a matching W configuration across the interior surface of the interior telescoping member 92 and the bottom of the side rails 90 as shown in FIG. 9.

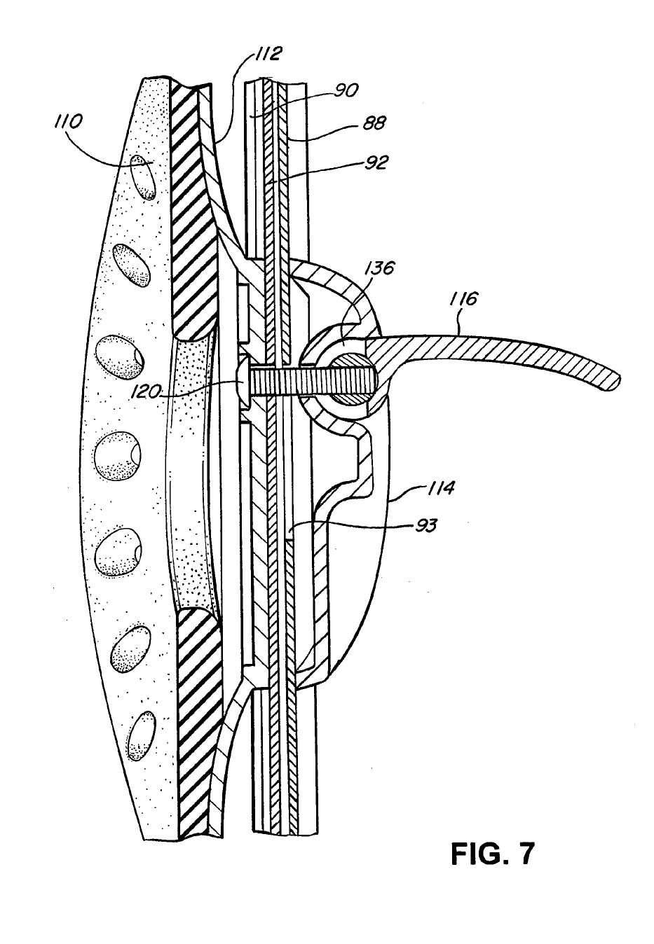

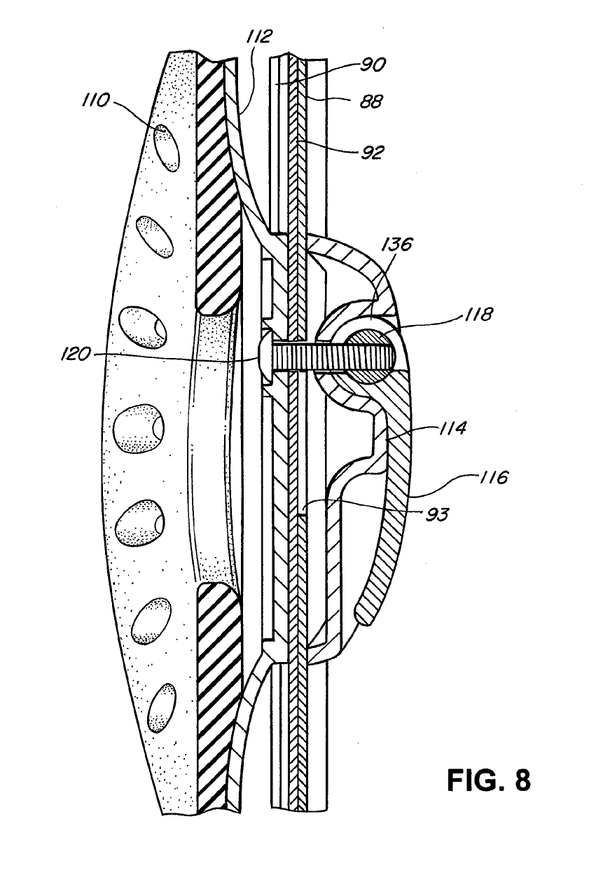

Referring to FIGS. 7 and 8, it can be appreciated that a connector such as the single bolt member 120, when operatively connected to the barrel nut 118 for mounting a movable control member 116 such as a pivotal lever, can permit both the exterior telescoping member 88 and the interior telescoping member 92 to relatively move within the limitation of the appropriate slots 93 and with such a movement, the thigh support member 70 can slide along the adjustable extension unit 68.

The movable control member 116 has a camming surface of such a dimension that when the control member 116 is in an open position, as shown in FIG. 7, in the form of a pivotal lever 116, the bolt 120 is extended to permit the sliding movement. However, when the pivotal lever 116 is closed as shown in FIG. 8, the bolt 120 is pulled to the right and clamps the external telescoping member 88 and the interior telescoping member 92 together so that the sliding contact base 126 of the concave paddle member 112 is pulled to compress and hold fast both the thigh support unit 70 and the adjustable extension unit 68 in a fixed position. As can be appreciated, the movable control member 116 can take other configurations such as a rotable worm gear, sliding wedges or other structures to provide the relatively small movement of the bolt member 120 for locking the adjustable extension unit 68 and the thigh support unit 70.

While the orthosis is shown as a hip orthosis, the present invention can be used to permit a simple adjustment of other body support units that are appended to a patient. For example, a spine brace that can be attached to the patient's waist and to the shoulders with an adjustable telescoping extension unit extending along the spinal column and a body support unit with a surface conforming to the back of the patient adjustably moved along the adjustable telescoping extension unit and lockable at a desired position to set both the back support unit location and length of the adjustable telescoping extension unit with one movable control member motion.

Those skilled in the art will appreciate that various adaptations and modifications of the just-described preferred embodiment can be configured without departing from the scope and spirit of the invention. Therefore, it is to be understood that, within the scope of the amended claims, the invention may be practiced other than as specifically described herein.

1. A hip orthosis comprising:
a hip engaging unit including a hip member for conforming to a waist of a user about the user's pelvis and a closure unit for securing the hip engaging unit on the waist to enable a lateral force to be applied to the user's pelvis opposite a surgical site;
a thigh engaging unit including a malleable thigh member for conforming to a thigh of a user, to engage a femur of the user on the surgical site side and on a distal side of the femur;
an adjustable extension unit connected to the hip engaging unit and the thigh engaging unit to enable adjustment to accommodate the size of the user; and
a thigh support unit connected to the adjustable extension unit at a position between the hip engaging unit and the thigh engaging unit to engage and laterally press the femur of the user, on the surgical side, whereby a force is translated through the femur and pelvis to push a head of the femur into the acetabulum of the user when the hip engaging unit and thigh engaging unit are fastened to the user.

2. The hip orthosis of claim 1 wherein the adjustable extension unit includes a pair of elongated members that overlap and are mounted for telescoping movement.

3. The hip orthosis of claim 2 wherein the adjustable extension elongated members have an approximately W cross sectional shape with an outer extension member capturing an interior extension member and the thigh support unit is slidably mounted on the extension members to releasably lock and hold the thigh support unit at a predetermined location along the extension members.

4. The hip orthosis of claim 3 wherein the thigh support member includes a flexible concave paddle member configured to interface with the surgical side surface of the leg below the surgical site to exert a force for pushing the head of the user's femur into the acetabulum of the user.

5. The hip orthosis of claim 4 wherein the concave paddle member has a central base for sliding contact with the adjustable extension unit, and adjacent side openings to enable respective outer edge panels of the paddle member to relatively move in relation to the central base to permit adjustment to a configuration of the user's leg.

6. The hip orthosis of claim 4 wherein the concave paddle member has a central base for sliding contact with the adjustable extension unit and a support housing mounting a movable control member that is operatively connected to the central base wherein movement of the control member locks the concave paddle member on the adjustable extension unit.

7. The hip orthosis of claim 6 where the movable control member is a pivotal lever member that forces the central base member into a pressurized contact with the adjustable extension unit to lock the concave paddle member and the adjustable extension unit outer and interior extension members together.

8. The hip orthosis of claim 7 wherein the central base, the support housing and the pivotal lever are interconnected by a connector member extending through openings in the adjustable extension unit whereby the adjustable extension unit is held in a fixed position when the pivotal lever is moved to force the central base to compress the adjustable extension unit together while locking the concave paddle member in a predetermined position on the adjustable extension unit to fit the user.

9. The hip orthosis of claim 1 wherein the malleable thigh member has a U configuration for partially encircling a user's thigh approximately above the user's knee joint with a flexible and adjustable closure member connected to a female receptacle member and a male prong locking member to enable the user to set and release the closure member at a predetermined fixed distance, when the male prong locking member is mounted in the female receptacle member, by compressing the male prong locking member.

10. The hip orthosis of claim 4 wherein the closure member includes hook and nap material and the U-shaped thigh malleable member is formed from plastic.

11.-13. (canceled)

14. The hip orthosis of claim 1 where the adjustable extension unit and the hip engaging unit are interconnected by a hip hinge unit that permits a flexion/extension adjustment and an abduction adjustment pivot member.

15. The hip orthosis of claim 1 further including compressed foam pads covering, respectively, the thigh support unit, the hip engaging unit and the thigh engaging unit and the adjustable extension unit has a slot opening to enable relative sliding engagement with the thigh support unit.

16. In an orthosis for supporting a body portion of a user, the improvement comprising:
a body support unit with a support surface for conforming to a body surface part of the user;
an adjustable extension unit slidably connected to the body support unit and releasably connected to the user including a pair of telescoping elongated members that are relatively movable to adjust a position of the body support unit for the user, wherein the body support unit has a base portion for slidable contact with the adjustable extension unit; and
a fastening member that locks the body support unit and the pair of telescoping elongated members to a fixed position.

17. The orthosis of claim 16 wherein the fastening member when moved to a locked position forces the base portion into a locking pressurized contact with the adjustable extension unit to prevent relative movement of the telescoping elongated members while locking the body support unit in a predetermined position on the adjustable extension unit.

18.-20. (canceled)

21. The orthosis of claim 17 wherein the body support unit includes a flexible concave paddle member configured to interface with a surgical side surface of a user's leg below a surgical site to exert a force for pushing the head of the user's femur into the acetabulum of the user.

22. the orthosis of claim 21 wherein the concave paddle member has a central base for sliding contact with the adjustable extension unit, and adjacent side openings to enable respective outer edge panels of the concave paddle member to relatively move in relation to the central base to permit adjustment to a configuration of the user's leg.

23. In an orthosis for supporting a body portion of a user, the improvement comprising:
an adjustable extension unit including an interior extension member and an outer extension member slidably mounted to capture the interior member, the extension members have an approximately W cross sectional shape to provide a width dimension larger than a thickness dimension, the interior member and the exterior member can be relatively moved to adjust a support position suitable to a size of the user; and
a fastener member to lock the interior and exterior extension members together at the size of the user.

24. The orthosis of claim 23 further including a body support unit mountable on the adjustable extension unit for contacting the body portion.

25. The orthosis of claim 24 where the body support unit is a concave paddle member of a flexible plastic material with a base that is configured to slide along the extension unit and to be locked to the extension unit by the fastener member.

26. The orthosis of claim 25 wherein the concave paddle member is formed of one of nylon and polypropylene and has adjacent side openings to enable respective outer edge panels of the concave paddle member to relatively move in relation to the base to permit adjustment to a configuration of the user's body and a resilient compression pad is adhered to the concave paddle member for direct contact with the user's body.
